(19) United States
(12) Patent Application Publication
(10) Pub. No.: US 2009/0229610 A1
(43) Pub. Date: Sep. 17, 2009

(54) Network Enabled Flow Generator

(75) Inventors: John David Oates, Stanhope Gardens (AU); Paulos Aklilu, Mount Druitt (AU); Michael Waclaw Colefax, Normanhurst (AU); Christophe Waterplas, Chiswick (AU); Michael James Hoade, Hammersmith (GB)

Correspondence Address: NIXON & VANDERHYE, PC, 901 NORTH GLEBE ROAD, 11TH FLOOR, ARLINGTON, VA, 22203, US

(73) Assignee: Resmed Ltd., Bella Vista (AU)

(21) Appl. No.: 11/989,234

(22) PCT Filed: Sep. 12, 2006
(86) PCT No.: PCT/AU2006/001343

§ 371 Date: Jan. 23, 2008

Related U.S. Application Data
(60) Provisional application No. 60/715,612, filed on Sep. 12, 2005.

(51) Int. Cl. A61M 16/00

(52) U.S. Cl. 128/204.21; 128/204.23

(57) ABSTRACT

A network enabled flow generator includes a continuous source of breathable gas for delivery to airways of a patient, and a process controller processing data relating to operations of the flow generator. A network interface communicates with the process controller and is configured to support a communications protocol. In this manner, patient data can be more easily accessed, and the flow generator can be more easily maintained via access through a network such as the Internet.

### CROSS-REFERENCES TO PRIORITY APPLICATION

This application claims the benefit of U.S. Provisional Application No. 60/715,612, filed Sep. 12, 2005, incorporated herein by reference in its entirety.

### BACKGROUND OF THE INVENTION

The present invention relates to a flow generator for ventilatory assistance and, more particularly, to a flow generator that is configured for communication and access via a network such as the Internet.

Non-Invasive Positive Pressure Ventilation (NIPPV) is a form of treatment for breathing disorders which can involve providing a relatively higher pressure of air or other breathable gas to the entrance of a patient's airways via a patient interface (e.g., a mask) during the inspiratory phase of respiration, and providing a relatively lower pressure or atmospheric pressure in the patient mask during the expiratory phase of respiration. In other NIPPV modes the pressure can be made to vary in a complex manner throughout the respiratory cycle. For example, the pressure at the mask during inspiration or expiration can be varied through the period of treatment.

Continuous Positive Airway Pressure (CPAP) treatment is commonly used to treat breathing disorders including Obstructive Sleep Apnea (OSA). CPAP treatment continuously provides pressurized air or other breathable gas to the entrance of a patient's airways via a patient interface (e.g., a mask) at a pressure elevated above atmospheric pressure, typically in the range 3-20 cm H₂O. CPAP treatment can act as a pneumatic splint of a patient's upper airway.

CPAP treatment can be in a number of forms, including the maintenance of a constant treatment pressure level, alternating between two different constant levels in synchronism with the inspiratory and expiratory phases of respiration (“bi-level CPAP”), and having an automatically adjustable and/or a computer controlled level in accordance with a patient's therapeutic needs. In all of these cases there is a need for control over the pressure of air or breathable gas supplied to the patient mask.

Breathable gas supply apparatus used in CPAP and NIPPV treatments broadly comprise a flow generator constituted by a continuous source of air or other breathable gas generally in the form of a blower driven by an electric motor. A pressurized supply of air or other breathable gas can also be used. The gas supply is connected to a conduit or tube, which is in turn connected to a patient interface (mask or nasal prong) which incorporates, or has in close proximity, a vent to atmosphere for exhausting exhaled gases, such as carbon dioxide.

The quality of care management a physician can provide to patients using flow generators relies not only on the quality of the data but also on the accessibility of that data. This is compounded in non-invasive ventilation where the volume of data further reduces its accessibility. This problem is of such a magnitude that many physicians ignore the data completely and rely simply on the patient's own report on their condition.

Existing solutions primarily rely on the mailing of various types of storage media, for example, smart media cards or the like, or on the use of paging systems. Another solution has been to directly connect the flow generator with a PC via a serial cable.

### BRIEF SUMMARY OF THE INVENTION

It would be desirable for a flow generator to enable a physician to access data in a timely fashion and thereby improve the quality of care given to a patient. Additional advantages could be attained if patients could be monitored remotely in real time. The patients would thus receive better management of their condition as a physician becomes better informed about the patients' condition, and they are better able to make decisions about appropriate care. Additionally, the more a patient can be educated about their own condition, the more likely it is that they will accept the treatment, and the more effective the treatment will become.

Use of a home-based flow generator would reduce the need to use sleep labs to assess a patient's condition. Currently, accessibility to sleep labs is limited, causing delays in patient prescriptions. Furthermore, the cost for performing the tests can be reduced by bypassing the critical resource. A home-based system would also provide more opportunity for chronically ill patients to be treated at home, thereby reducing costs and putting the patients in a comfortable environment.

In an exemplary embodiment of the invention, a flow generator is configured for communication and access via a network. The flow generator includes a continuous source of breathable gas for delivery to airways of a patient, and a process controller processing data relating to operations of the flow generator. A network interface supporting an Internet protocol is coupled with the process controller. In a preferred arrangement, the network interface includes a TCP/IP stack or a TCP/IP stack and an HTTP web server. The process controller may include a memory or other storage medium that stores the data relating to operations of the flow generator. In another arrangement, the process controller is provided with a client e-mail application or Mail User Agent (MUA) for generating an e-mail communication deliverable via the network interface. A user input interface may be provided to communicate with the process controller for receiving patient derived data. In this context, the flow generator may include a display screen, and, further, the user input interface may include a keyboard, mouse, or other suitable input device. In an alternative arrangement, the user input interface may include a connector attachable to an external device.

In another exemplary embodiment of the invention, a method enables communicating data relating to operations of a flow generator over a global network. The method includes the steps of storing the data relating to operations of the flow generator, and enabling access to the data via a remote computer with Internet access and using a web browser. In this context, the storing step may be practiced by storing time-based traces of the data. The enabling step may be practiced by enabling access to real time data when the flow generator is in use. The remote computer may also review and modify flow generator operational settings via communication with the process controller through the network interface.

In another arrangement, the method may include a step of generating an e-mail communication deliverable via the network interface. In this context, the step of generating an e-mail communication may be further practiced by sending the e-mail communication via the network interface to at least one of a physician, an SMS gateway for relay into a mobile phone network, an Internet maintained database, and a sleep lab service center with Internet connectivity. Alternatively or additionally, the step of generating an e-mail communication may be further practiced by sending the e-mail communication via the network interface to at least one of a service center to notify a fault occurrence detected by the process controller and an Internet connected server that logs usage/compliance and efficacy data to a database. Additionally the flow generator may allow for communications with a remote person in real time such as a nurse or help desk. The communications may be in the form of text using Internet Relay Chat (IRC) (e.g. like an online chat session), sound (e.g. Voice over IP), or video using an built-in webcam.

The storing step preferably includes storing at least one of images and programs relating to the operations of the flow generator, and the method preferably further comprises referencing the images and/or programs in web pages delivered to the web browser on the remote computer, the images and programs being loadable into the web browser using standard HTTP or other suitable protocol.

In another arrangement, the enabling step may be practiced by connecting the flow generator to a health facility local area network. In this context, the method may further include the steps of monitoring the data via a central workstation at the health facility, and generating alerts based on the data.

The enabling step may be practiced by connecting the flow generator with a user's home network. The home network may include a home computer running a software program for analyzing the data relating to operations of the flow generator, and the enabling step is practiced by enabling the home computer to analyze the data and access information on patient care based on the analyzed data. In another arrangement, the enabling step may be practiced by connecting the flow generator to a local area network that includes an Internet gateway, the flow generator periodically communicating with an Internet connected server to determine if a later version of the flow generator operating software is available for download, and if so, downloading the operating software. With the network interface including a TCP/IP stack and a HTTP web server, the enabling step may be practiced by connecting the flow generator to a local area network that includes a service computer effecting flow generator maintenance and support, the service computer utilizing tools via the TCP/IP stack to gain access to the internal operation of the flow generator.

### BRIEF DESCRIPTION OF THE DRAWINGS

These and other aspects and advantages of the present invention will be described in detail with reference to the accompanying drawings, in which:

FIG. 1 is a perspective view of an exemplary flow generator; and

FIGS. 2-7 are schematic diagrams showing exemplary usage scenarios with the flow generator of the present invention.

### DETAILED DESCRIPTION OF PREFERRED EMBODIMENTS

### Flow Generator

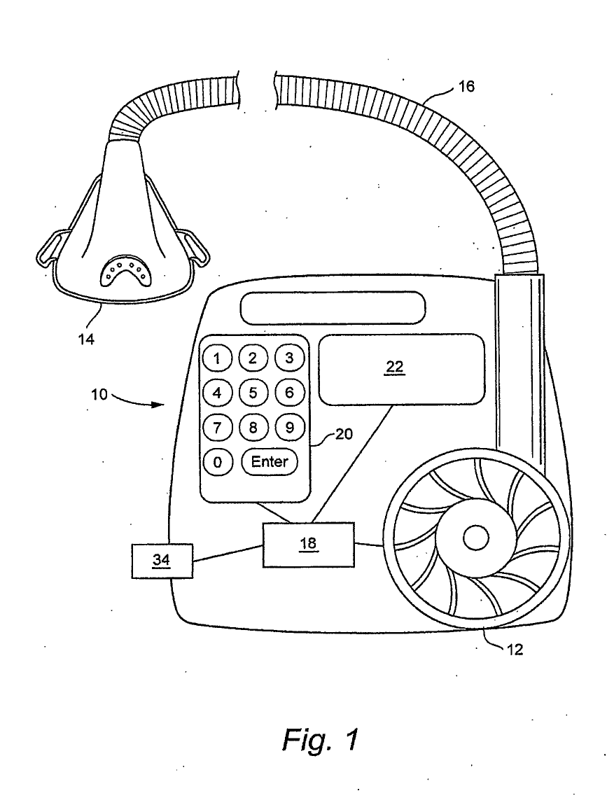

The concepts of the present invention are suitable for any flow generator providing NIPPV and/or CPAP treatment, including but not limited to flow generators having motor controlled pressure regulation or valve pressure regulation. An exemplary flow generator structure will be described with reference to FIG. 1 for purposes of explanation.

A flow generator 10 includes a motor 12 that provides a supply of pressurized air for the administration of NIPPV and/or CPAP treatment. The pressurized air is delivered to a patient via a patient interface 14. An air delivery conduit 16 is coupled between the flow generator 10 and the patient interface 14. The patient interface 14 may have any suitable configuration as is known in the art, e.g., full-face mask, nasal mask, oro-nasal mask, mouth mask, nasal prongs, etc. Furthermore, the patient interface 14 also encompasses both vented and non-vented masks and dual limb mask systems. A process controller 18 controls the operations of the flow generator. The flow generator may comprise a user interface unit 20 to allow information input and a display unit 22 to display output information.

The flow generator 10 may also comprise sensors to determine the delivered pressure or flow through the device and to detect breathing (respiration) on the device. For example the device may use an in-line flow sensor to measure flow directly or use pressure transducers to measure the pressure drop along the air delivery conduit or across a restriction in the delivery conduit. Alternatively, the flow may be estimated using the motor speed and current as described in U.S. Pat. No. 5,740,795 (Brydon et al.), U.S. Pat. No. 6,237,593 (Brydon et al.), and U.S. provisional application 60/624,951, all having the same assignee as the present invention.

Network Interface

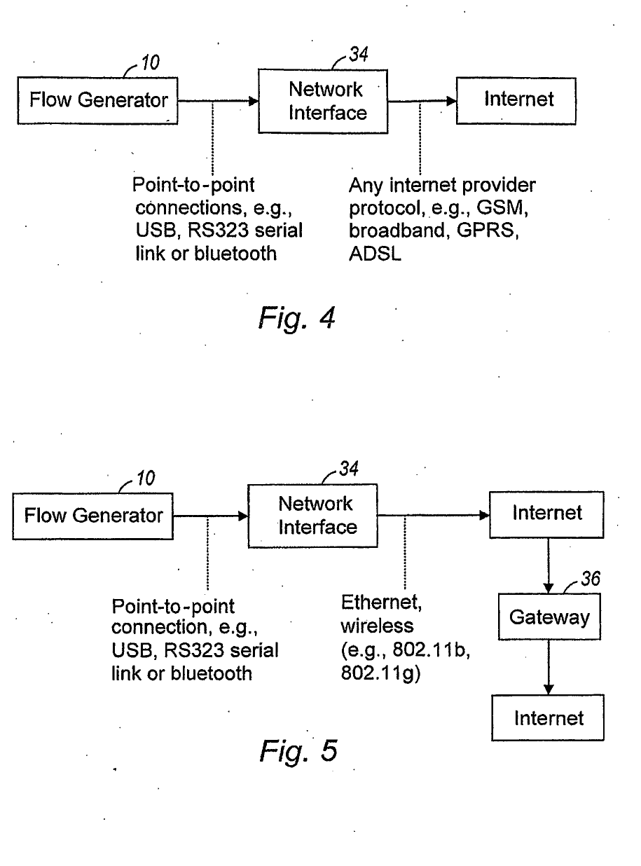

The flow generator 10 is also provided with a network interface 34 communicating between the process controller 18 and the output appropriate to the given embodiment via a communications protocol. Although any suitable communications protocol may be included, in a preferred embodiment, the communications protocol comprises a TCP/IP stack with or without an HTTP (hypertext transfer protocol) web server. The network interface 34 allows the flow generator 10 to be connected to the Internet, or any other potentially-global network, using any connection that can support the communications protocol, including, but not limited to, universal serial bus (USB), Bluetooth, RS232 serial link, and the like for point to point connections between the flow generator 10 and the network interface 34, and ethernet or wireless network (e.g., 802.11b, 802.11g, etc.) and the like for connection to the local area network (LAN). For example, in the home, the flow generator 10 may be connected via a USB, Bluetooth or RS232 serial link to the network interface 34 and then to the Internet via any public internet transport protocol such as GSM, GPRS, Broadband or ADSL. See FIG. 4.

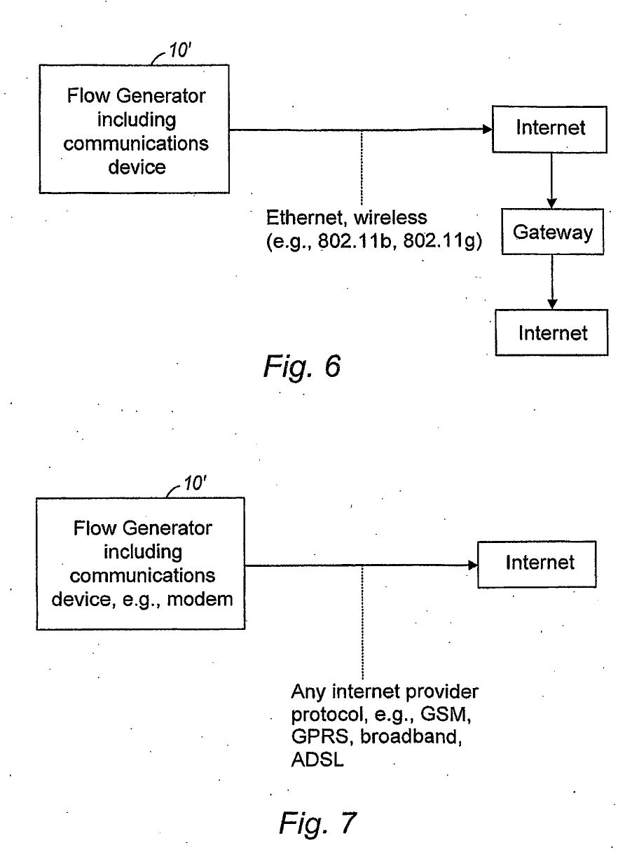

In another embodiment, particularly suitable in a hospital, the flow generator 10 may be connected to the network interface 34 in a similar manner as described above, and the network interface 34 connects to a LAN via an Ethernet or wireless (e.g. 802.11b or 802.11g) connection, and then an Internet gateway 36 links the LAN to the internet. See FIG. 5. In a further embodiment, the network communications device is integrated into the flow generator 10′ such that a direct connection to a local area network may be enabled using Ethernet or wireless connections such as 802.11b or 802.11g. In this embodiment, the network interface 34 is not required. See FIG. 6. The flow generator 10′ may also comprise a network communications device such as a modem that allows direct connection to the Internet. In this case, the network interface 34 is incorporated within the flow generator 10. See FIG. 7.

Once connected, the flow generator 10 can be accessed remotely by any computer with access to the Internet using a standard web browser or any interface program with socket connectivity, including both generic or specific interface programs. The web browser can be used to display time based traces of both stored data and data as it is acquired, or to display and set both text and numeric values on the flow generator 10. Examples of data types that may be communicated from the flow generator include: (1) events such as alarms, alerts, system diagnostic information, modifications of settings and usage data such as commencement, interruption or completion of use; (2) historical recorded time-based traces such as patient flow and mask pressure (static); (3) real-time time-based traces such as patient flow and pressure (dynamic); (4) identification information such as patient information, equipment identification and information; (5) communications and multimedia such as video, sound and text (for example, the system may provide a video on how to use the device or to fit the mask or may include a web cam to view the position of the patient); etc. Of course, those of ordinary skill in the art will appreciate still other data types suitable for processing by the flow generator of the invention, and the invention is not necessarily meant to be limited to the described examples.

The process controller 18 may further include a client e-mail application or Mail User Agent (MUA) for generating an e-mail communication deliverable via the network interface 34. E-mail may be sent to any e-mail address, such as to a trading positions an SMS gateway for relay into a mobile phone network, an Internet-maintained database, a sleep lab service center with Internet connectivity, and the like. The e-mail may be solicited. For example, the message may be generated as a as a response to a request or instruction received through a network connection, the request relating to treatment and/or patient condition data, the status of the flow generator (e.g., whether a fault has occurred), etc. The instruction may include, for example, at least a request for information to be included in the e-mail and an address to which the e-mail should be sent. Alternatively or in addition, the e-mail may be unsolicited. For example, an automatically generated message may be sent periodically and/or at a predetermined time (e.g., hourly, daily/nightly, after each treatment session, etc.) to report treatment and/or patient condition data, the status of the flow generator, or the like. The e-mail also may be sent when a predefined event occurs (e.g., upon an indication of a certain patient condition, such as, for example, when the patient suffers a severe apnea; when a fault in the flow generator is detected; etc.).

Protocols

As would be apparent to those of ordinary skill in the art, the TCP/IP stack includes support for any standard protocol used in local area networks. Examples of such protocols include:
TCP and UDP Sockets to interact with custom applications or applets running on computers connected to the network
Real Time Protocol (RTP), Real Time Streaming Protocol (RTSP), and Real Time Control Protocol (RTCP) to permit the transmission of multiple data streams in a time coherent fashion
ICMP to support ping
Simple Mail Transfer Protocol (SMTP) to permit transmission of e-mail
Domain Name Service (DNS) to permit the use of web URLs rather than just IP numbers
Dynamic Host Configuration Protocol (DHCP) to allow the flow generator to automatically be allocated an IP address
File Transfer Protocol (FTP) to allow the dynamic remote upgrading of flow generator software
Internet Relay Chat (IRC) to allow direct two-way e-mail communications in real time.

The process controller 18 of the flow generator 10 also has the ability to store images and applets that can be referenced in the web pages that it delivers to the web browsers. These images and applets are then able to be loaded into the browser from the flow generator 10 using standard HTTP.

Exemplary Usage Scenarios

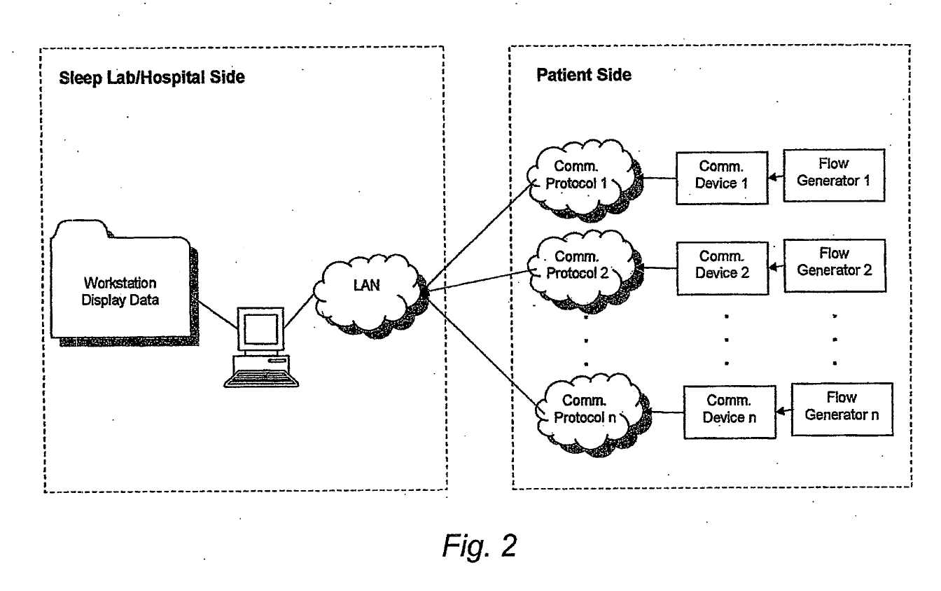

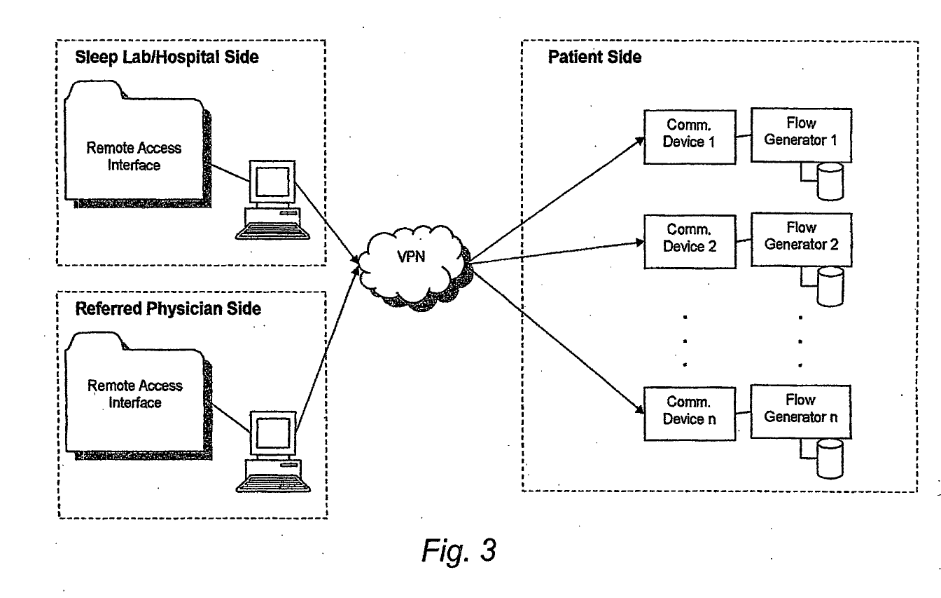

Exemplary usage scenarios of the present invention are illustrated in the schematic diagrams of FIGS. 2 and 3. With the network enabled flow generator 10 of the invention, the flow generator 10 can be connected to a local area network (LAN) of a health facility such as a sleep lab or hospital. In this context, data relating to the operation of the flow generator processed by the process controller 18 can be monitored at a central work station manned by sleep technicians or nurses. New flow generators added to the network are automatically added to the local display software. Alerts for various conditions can also be transmitted to the work station(s) to notify the technicians. This connection also enables remote access to the flow generators by a physician, permitting the physician to care for a patient even when they are at a remote location. Access would preferably be via a virtual private network (VPN), which would assist in providing secure access to patient data, and the security of the data itself. This scenario would also facilitate obtaining a second opinion from another physician who would only need a computer with access to the Internet and the ability to connect to the remote network using a VPN to gain access to the patient's flow generator, even while it is in use. It will be appreciated that those of ordinary skill in the art may use other security mechanisms in place of, or in addition to, a VPN to provide for the security of patient data.

The flow generator may be connected in a patient's home using broadband Internet connection or wireless, such as GSM or GPRS, or the like. This scenario permits a physician to view data on the flow generator at any time from any location. Preferably, access would be via a VPN. The broadband connection could include, for example, a cable modem, a DSL modem over the patient's phone line, high-speed wireless access, or any other high-speed connection that may include either, or both of, a physical or a wireless connection. A broadband connection is preferred over a non-broadband connection for performance reasons, though it is to be appreciated that the flow generator could accommodate a slower connection.

The flow generator located in the patient's home may also be connected to the patient's own home-based network. This connection would allow the patient access to data on their own care without installing any software other than a standard web browser. The patient interface can be further enhanced by including integration with both automated and manned Internet-based help desk facilities. Such facilities can include video on care and maintenance of the flow generator, and information subjects such as choosing and fitting masks and the like.

In another exemplary usage scenario, the flow generator may be connected to a LAN including a mail server. The flow generator could then initiate an e-mail to a service center to notify them of a fault that the flow generator has self-detected. This permits the service center to initiate repair or replacement rather than waiting for the user/clinician to do so. Alternatively or additionally, the flow generator can initiate an e-mail to an Internet connected server that logs usage/compliance and efficacy data to a database. This data can include traditional sleep apnea data as well as data pertinent to sleep labs and hospitals such as the number of patients that have used a particular flow generator and ventilation levels of various patients.

The flow generator may be connected to a LAN that includes an Internet gateway so that the flow generator can periodically poll an Internet connected server to determine if a later version of the flow generator software, or a component of the software, is available for download. If so, the flow generator can begin a process to upgrade its own software at an appropriate time.

The flow generator may be connected to a LAN that includes the flow generator software developer's computer, enabling the developer to then use tools that utilize the TCP/IP stack to gain access to the internal operation of the flow generator. This access provides a rich data set that aids the developer in quickly diagnosing any faults in the software while under development.

The network interface may additionally comprise a user input interface communicating with the process controller for receiving patient derived data. A software program or the like may request data to be input by the user possibly via a keyboard and display screen or other user input device. Alternatively, the user input interface may include a connector attachable directly to an external device. These patient derived signals can then be transmitted with any of the flow generator time based signals in a time coherent fashion using any of the noted connection scenarios. Signals may include SaO₂, partial CO₂, EMG, EOG, EEG and the like.

It may be desirable to provide an operating system in the flow generator that safeguards against malicious attack. To ensure that the patient receives the correct therapy it is necessary to ensure that within every predefined sample period:

1. Pressure and flow signals are converted to digital signals

2. The appropriate algorithms are executed using the acquired digital signals

3. The various alarm conditions are evaluated and the appropriate actions taken in the case of one of the conditions being true

4. The motor control signal is updated

Failure to execute these steps within the predefined sample period will result in modified behavior of the flow generator, and in the worst case prevent it from raising an alarm or terminating therapy when it should.

An HTTP server (or more generally a TCP/IP stack) requires the microprocessor to do some processing for every request that is received. If someone with malicious intent wanted to interfere with the flow generator operation, one possible cause of action would be to generate requests of the server at a rate faster than it could respond. This is known as a Denial of Service attack, however it is usually associated with Internet services being denied, not medical therapy. If the flow generator software is not designed to cope with this problem, the important steps listed above may not be performed within the time required.

Note that the system preferably includes a watchdog component, which monitors the microprocessor to ensure that it meets the above conditions and will cause it to reset if it does not. This component may not solve the issue, however, as the malicious requests could continue, resulting in the flow generator being repeatedly reset.

A solution has been to design the flow generator software around two separate processing loops. One loop simply executes as frequently as it can, restarting every time it reaches the end. The second loop is executed on an interrupt that occurs at the required sample period. This loop contains all the processing elements that must be performed within every sample period as described above.

As all the HTTP server and TCP/IP stack processing is executed on the first loop, which is interrupted every time a new sample period occurs, it is impossible for any behavior of the server or the stack to interfere with the processing of the second loop. The processing of the first loop is also protected by ensuring that the TCP/IP stack and HTTP server will only respond to one request on any one execution of the first loop. If the requests of the TCP/IP stack are generated faster than the system can respond, it simply drops the requests. Whilst this does not stop the malicious perpetrator from successfully preventing the TCP/IP services of the flow generator, it does prevent the loss of any service to the patient.

One of the more challenging aspects of implementing the invention is to design and implement a lightweight TCP/IP stack and HTTP server that do not use any interrupts. In one embodiment of the invention, a TCP/IP stack uses each layer of the stack to handle just one request at any one time. Before starting to process a new request, the element in the stack has to first check that the previous element has been fully processed by the adjacent level in the stack. This design guarantees that each request will either be fully processed or dropped completely. It also ensures that a predetermined amount of flow generator memory and processor time are required to complete requests irrespective of the volume/speed of the TCP/IP traffic.

A lightweight HTTP server suitable for a flow generator may be implemented if the flow generator uses a simple interpreter. Processing modules within the flow generator define commands that have a signature, which is a template that will match certain ASCII strings. When a command is found with a suitable signature, it is invoked and returns an ASCII string. In the case of an HTTP server, the commands are derived from the URL posted by the client. If a command with a suitable signature cannot be found, a default command is executed. Each command returns an ASCII string that contains the HTML to be displayed in the browser. The default command returns an HTML page that informs the user of an error. The server is further enhanced by having a command that is able to read files from the flow generator's persistent data store. These files can include html files, graphics files, sound files, video files, or a file in any format that is understood by standard browsers. The files can also include Java applets which are used to display dynamically changing data within a web browser. The applets gain their data by opening a socket with the flow generator on a dedicated TCP/IP port number.

### CONCLUSION

With the network enabled flow generator of the invention, physicians can access more data in a timely fashion to improve quality of care given to a patient. Additionally, the system will enable physicians to monitor patients in real time without needing to be in the same location as the patient. Additionally, physicians can refer patients to other physicians located anywhere in the world if necessary. For the patient's perspective, the system enables better management of their condition since the physician becomes better informed about a patient's condition, and the physician is able to make better decisions about appropriate patient care. The patient will be better informed, and the more a patient can be educated about their own condition, the more likely it is that they will accept the treatment and the more effective the treatment will become. Use of a home-based flow generator would reduce the need to use sleep labs to assess a patient's condition. Currently, accessibility to sleep labs is limited, causing delays in patient prescriptions. Furthermore, the costs for performing the tests can be reduced by bypassing the critical resource. The system will also provide more opportunity for chronically ill patients to be treated at home, thereby reducing costs and putting the patient in a more comfortable environment.

While the invention has been described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention.

1. A network enabled flow generator comprising:
a continuous source of breathable gas for delivery to airways of a patient;
a process controller to communicate with the gas source and to process data relating to operations of the flow generator; and
a network interface supporting a communications protocol, the network interface being configured to communicate with the process controller.

2. A network enabled flow generator according to claim 1, wherein the communications protocol comprises one of a TCP/IP stack or a TCP/IP stack and an HTTP web server.

3. A network enabled flow generator according to claim 1, wherein the process controller comprises a memory to store the data relating to operations of the flow generator.

4. A network enabled flow generator according to claim 1, wherein the process controller comprises a client email application or Mail User Agent (MUA) to generate an e-mail communication deliverable via the network interface.

5. A network enabled flow generator according to claim 1, further comprising a user input interface to communicate with the process controller, the user input interface receiving patient derived data.

6. A network enabled flow generator according to claim 5, further comprising a display screen, wherein the user input interface comprises a keyboard.

7. A network enabled flow generator according to claim 5, wherein the user input interface comprises a connector attachable to an external device.

8. A network enabled flow generator according to claim 1, wherein the network interface is connected to a LAN via an Ethernet or wireless connection, and wherein an Internet gateway links the LAN to the internet.

9. A network enabled flow generator according to claim 1, wherein the network interface is incorporated within the flow generator.

10. A method of communicating data relating to operations of a flow generator for a patient over a global network, the method comprising:
storing the data relating to operations of the flow generator; and
enabling access to the data via at least one remote computer with Internet access using a web browser.

11. A method according to claim 10, wherein the storing step is practiced by storing time-based traces of the data.

12. A method according to claim 10, wherein the enabling step is practiced by enabling access to real time data when the flow generator is in use.

13. A method according to claim 10, further comprising enabling the remote computer to review and modify flow generator operational settings via communication with a process controller through a network interface.

14. A method according to claim 10, further comprising generating an e-mail communication deliverable via a network interface.

15. A method according to claim 14, wherein the step of generating an e-mail communication is further practiced by sending the e-mail communication via the network interface to at least one of a physician, an SMS gateway for relay into a mobile phone network, an Internet maintained database, and a sleep lab service center with Internet connectivity.

16. A method according to claim 14, wherein the step of generating an e-mail communication is further practiced by sending the e-mail communication via the network interface to at least one of a service center to notify a fault occurrence detected by a process controller and an Internet connected server that logs usage/compliance and efficacy data to a database.

17. A method according to claim 10, wherein the storing step comprises storing at least one of images and programs relating to operations of the flow generator, the method further comprising referencing the images and/or programs in web pages delivered to the web browser on the remote computer, the images and applets being loadable into the web browser using standard HTTP.

18. A method according to claim 10, wherein the enabling step is practiced by connecting the flow generator to a health facility local area network.

19. A method according to claim 18, further comprising monitoring the data via a central workstation at the health facility, and generating alerts based on the data.

20. A method according to claim 10, wherein the enabling step is practiced by connecting the flow generator with a user's home network.

21. A method according to claim 20, wherein the home network includes a home computer running a software program for analyzing the data relating to operations of the flow generator, the enabling step comprising enabling the home computer to analyze the data and access information on patient care based on the analyzed data.

22. A method according to claim 10, wherein the enabling step is practiced by connecting the flow generator to a local area network that includes an Internet gateway, the flow generator periodically communicating with an Internet connected server to determine if a later version of flow generator operating software is available for download, and if so, downloading the operating software.

23. A method according to claim 10, wherein the communications protocol comprises one of a TCP/IP stack or a TCP/IP stack and an HTTP web server, and wherein the enabling step is practiced by connecting the flow generator to a local area network that includes a service computer effecting flow generator maintenance and support, the service computer utilizing tools via the TCP/IP stack to gain access to internal operation of the flow generator.

24. A method according to claim 10, wherein the enabling step comprises running a software program using at least first and second processing loops, executing the first processing loop as frequently as possible, and executing the second processing loop on an interrupt that occurs at a sample period.

25. A method of communicating data relating to operations of a medical device over a global network, the method comprising:
storing the data relating to operations of the medical device; and
enabling access to the data via at least one remote computer with Internet access using a web browser.

26. A network enabled medical device comprising:
a medical device component to act on a patient; a process controller to process data relating to operations of the medical device; and
a network interface supporting a communications protocol, the network interface being configured to communicate with the process controller.

27. A network enabled flow generator comprising:
a continuous source of breathable gas for delivery to airways of a patient;
a process controller to communicate with the gas source and to process data relating to operations of the flow generator; and
a network communications device integrated into the flow generator, the network communications device being configured to communicate with the process controller, wherein a direct connection to a local area network may be enabled using an Ethernet or wireless connection.

28. A network enabled flow generator comprising:
a continuous source of breathable gas for delivery to airways of a patient;
means for processing data relating to operations of the flow generator; and
means for enabling access to the data via a remote computer with Internet access using a web browser.

29. A method according to claim 14, wherein the step of generating an e-mail communication is further practiced by automatically generating the e-mail communication in response to a predefined event and/or at a predefined time or time interval.

30. The method according to claim 29, wherein the predefined event corresponds to a detection of a fault and/or a condition of the patient.

31. The method according to claim 29, wherein the predefined time interval is one or more of hourly, daily, nightly, or after a therapy session.

32. The method according to claim 14, wherein the step of generating an e-mail communication is practiced in response to an instruction received via the network interface.

33. The method according to claim 32, wherein the e-mail communication includes information corresponding a condition of the patient and/or a status of the flow generator.

34. The method according to claim 32, wherein the instruction includes a request for information and/or an address to which the e-mail message will be sent.

35. The network enabled flow generator according to claim 27, wherein the network communications device is further operable to send a message, the message being automatically generated in response to a predefined event and/or at a predefined time or time interval.

36. The network enabled flow generator according to claim 35, wherein the predefined event corresponds to a detection of a fault and/or a condition of the patient.

37. The network enabled flow generator according to claim 35, wherein the predefined time interval is one or more of hourly, daily, nightly, or after a therapy session.

38. The network enabled flow generator according to claim 27, wherein the network communications device is further operable to send a message generated by the process controller in response to an instruction received via the network interface.

39. The network enabled flow generator according to claim 38, wherein the message includes information corresponding a condition of the patient and/or a status of the flow generator.

40. The network enabled flow generator according to claim 38, wherein the instruction includes a request for information and/or an address to which the message will be sent.
